(19) United States
(12) Patent Application Publication
(10) Pub. No.: US 2026/0224351 A1
(43) Pub. Date: Aug. 6, 2026

(54) ACCOMMODATING INTRAOCULAR LENSES AND ASSOCIATED ASSEMBLIES AND METHODS

(71) Applicant: Shifamed Holdings, LLC, Campbell, CA (US)

(72) Inventors: Davis Engelman, Los Gatos, CA (US); Juan Diego Perea, San Jose, CA (US); Zoran Milanovic, Campbell, CA (US); Owen Raybould, Sunnyvale, CA (US); Tom Saul, Portland, OR (US)

(21) Appl. No.: 19/149,251

(22) PCT Filed: Jan. 26, 2024
(86) PCT No.: PCT/US2024/013205

§ 371 Date: Jul. 18, 2025

Related U.S. Application Data
(60) Provisional application No. 63/481,995, filed on Jan. 27, 2023.

(51) Int. Cl. A61F 2/16

(52) U.S. Cl. CPC A61F 2/1624; A61F 2002/1682; A61F 2002/169; A61F 2002/16902; A61F 2250/0063

(57) ABSTRACT

An accommodating intraocular lens (AIOL) can include a fixed power lens and a base. The base can include an adjustable power lens and define a lens-receiving area configured to receive the fixed power lens. A change to the fixed power lens can cause an adjustment to an optical power of the adjustable power lens. In some embodiments, the fixed power lens can be rotated within the lens-receiving area relative to the base to cause the adjustment to the optical power. Additionally, or alternatively, the fixed power lens can be replaced with a second non-accommodating or other lens to cause the adjustment to the optical power. The second fixed power lens can have one or more dimensions that are greater or less than corresponding dimensions of the fixed power lens.

### CROSS-REFERENCE TO RELATED APPLICATION(S)

The present application claims priority to U.S. Provisional App. No. 63/481,995, filed Jan. 27, 2023, and incorporated herein by reference in its entirety.

### TECHNICAL FIELD

The present technology relates to accommodating intraocular lenses (AIOLs) and methods of implanting and assembling the same.

### BACKGROUND

Cataracts can affect a large percentage of the worldwide adult population with clouding of the native crystalline lens and resulting loss of vision. Patients with cataracts can be treated by native lens removal and surgical implantation of a synthetic intraocular lens (IOL).

Worldwide, there are millions of IOL implantation procedures performed annually. In the U.S., there are 3.5 million cataract procedures performed, while worldwide there are over 20 million annual procedures performed.

Although IOL implantation procedures can be effective at restoring vision, conventional IOLs have several drawbacks. For example, many prior IOLs are not able to change focus as a natural lens would (known as accommodation). Other drawbacks of conventional IOLs include refractive errors that occur after implantation and require glasses for correcting distance vision, or in other cases the IOLs can be effective in providing good far vision, but patients need glasses for intermediate and near vision.

Several multi-focal IOLs have been developed to address these drawbacks, but they too can have drawbacks. For example, although multi-focal IOLs generally perform well for reading and distance vision, in at least some instances such multi-focal IOLs may cause significant glare, halos, reduced contrast sensitivity, and other visual artifacts.

AIOLs have been proposed to provide accommodative optical power in response to the distance at which a patient views an object. However, such AIOLs are generally still in development and have different drawbacks. For example, prior AIOLs can provide insufficient accommodation after implantation or produce suboptimal refractive correction of the eye. The amount of accommodation of the prior AIOLs can also decrease after implantation in at least some instances. The prior AIOLs can also be too large to be inserted through a small incision of the eye and may require the incision to be somewhat larger than would be ideal. Also, at least some of the prior AIOLs can be unstable when placed in the eye, which can lead to incorrect accommodation and other errors.

Improved implantable intraocular lenses that accommodate with the natural mechanisms of controlling focusing of the eye that overcome at least some of the above deficiencies would be desirable. Ideally, such improved AIOLs would provide increased amounts of accommodation when implanted, provide refractive stability, introduce few if any perceptible visual artifacts, and allow the optical power of the eye to change from far vision to near vision in response to the distance of the object viewed by the patient.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIGS. 1A and 1B are perspective views of an AIOL configured in accordance with embodiments of the present technology.

FIG. 1C is a cross-section of the AIOL taken along line 1C-1C of FIG. 1B.

FIGS. 1D and 1E are perspective views illustrating rotation of a fixed lens of the AIOL of FIG. 1A relative to a base lens of the AIOL of FIG. 1A, in accordance with embodiments of the present technology.

FIGS. 2A and 2B are top cross-sections of another AIOL configured in accordance with embodiments of the present technology.

FIGS. 3A and 3B are side cross-sections of a further AIOL configured in accordance with embodiments of the present technology.

FIG. 4A is a perspective view of an AIOL having a removable annular cartridge and configured in accordance with embodiments of the present technology.

FIG. 4B is a side cross-sectional view of a cartridge structure of the AIOL of FIG. 4A taken along section line 4A-4A of FIG. 4A.

FIG. 4C is the side cross-sectional view of the cartridge structure of FIG. 4B and a standard lens configured in accordance with embodiments of the present technology.

FIG. 4D is a side cross-sectional view of another cartridge structure configured in accordance with embodiments of the present technology.

### DETAILED DESCRIPTION

The present technology is directed to AIOLs and methods for making and using such devices. In many of the embodiments disclosed herein, the AIOLs include a base and a fixed power lens configured to removably connect to the base. The base can define an adjustable lens and a lens-receiving area configured to receive the fixed power lens. Both the adjustable power lens and the fixed power lens can have, define, and/or otherwise provide one or more optical characteristics including an optical power, a depth of field, and/or a depth of focus. The adjustable power lens can have one or more dynamically adjustable ranges of optical characteristics, such as a dynamically adjustable range of optical powers and/or a dynamically adjustable range of depths of field. The fixed lens can have one or more fixed optical characteristics, such as a fixed optical power, a fixed depth of field, and/or a fixed depth of focus. The optical characteristics of the adjustable power lens can be adjusted, at least in part, in response to a change in contact between the fixed power lens and the base (e.g., a change to a position and/or orientation of the fixed lens relative to the base). Allowing the optical characteristics provided by the adjustable power lens to be adjusted in this manner is expected to improve the adjustability and/or patient-specificity of the optical correction provided by the AIOL.

The change in contact between the fixed power lens and the base can be achieved using a number of different mechanisms. In some embodiments, for example, the fixed power lens can be positioned within the lens-receiving area defined by the base and rotated relative to the base to change the range of dynamic adjustability provided by the adjustable power lens. Additionally, or alternatively, the fixed power lens can be removed and replaced with a second, different fixed power lens to change the range of dynamic adjustability provided by the adjustable power lens. In these and other embodiments, the change to the fixed lens can alter or adjust a sensitivity of the AIOL to external forces, such as compressive forces from an eye (e.g., a capsular bag) of the patient, which is expected to improve and/or increase the accommodative response of the AIOL. In at least some embodiments, the fixed lens can be rotated, removed replaced, and/or otherwise changed in vivo, including while the AIOL is implanted within an eye of a patient.

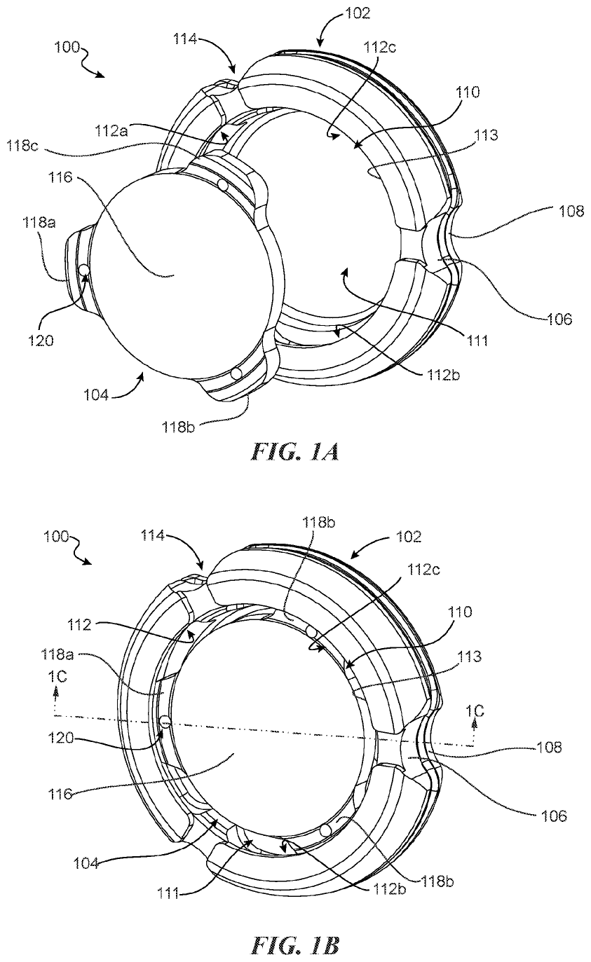

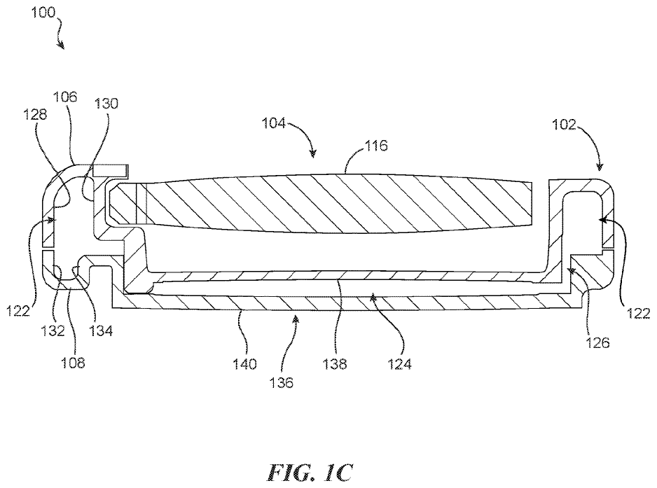

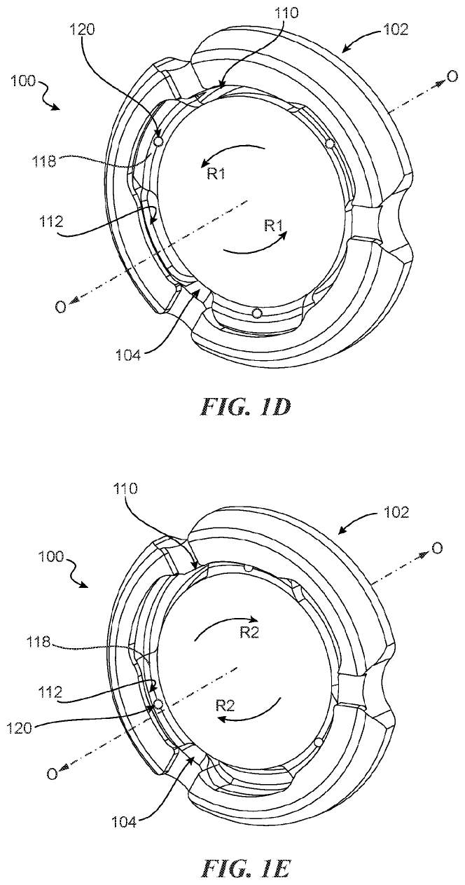

Specific details of various embodiments of the present technology are described below with reference to FIGS. 1A-4D. Although many of the embodiments are described below with respect to AIOLs and associated methods, other embodiments are within the scope of the present technology. Additionally, other embodiments of the present technology can have different configurations, components, and/or procedures than those described herein. For instance, AIOLs configured in accordance with the present technology may include additional elements and features beyond those described herein, or other embodiments may not include several of the elements and features shown and described herein.

As used herein, the use of relative terminology, such as “about,” “approximately,” “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

FIGS. 1A and 1B are perspective views of an adjustable intraocular lens 100 (“AIOL 100”) configured in accordance with embodiments of the present technology. The AIOL 100 can include an adjustable lens structure or base 102 (“base 102”) and a fixed lens 104. The base 102 can include a first or anterior component 106 and a second or posterior component 108, which can be coupled to one another to form the base 102. The first component 106 can define a concavity or lens-receiving area or volume 110 (“volume 110”) configured to receive at least a portion of the fixed lens 104. The volume 110 can include a chamber or cavity 111 having an opening 113. The volume 110, the cavity 111, and/or the opening 113 can have one or more dimensions (e.g., diameter, circumference, depth, volume, etc.) that correspond to one or more dimensions of the fixed lens 104, such that the fixed lens 104 can be positioned within the volume 110 by inserting the fixed lens 104 through the opening 113 and into the cavity 111. In some embodiments, the volume 110 can include one or more features configured to secure the fixed lens 104 within the volume 110 and/or otherwise prevent, or at least partially prevent, the fixed lens 104 from inadvertently leaving the volume 110 once the fixed lens 104 is positioned therein. In the illustrated embodiment, for example, the volume 110 includes one or more groove or slots 112 (individually identified as a first slot 112a, a second slot 112b, and a third slot 112c in FIGS. 1A and 1B) extending radially outward toward an outer perimeter of the base 102, and each of the slots 112 is configured to receive a respective portion of the fixed lens 104.

In some embodiments, the base 102 can include flow-through features 114 that enhance the rate and ease with which Ophthalmic Viscosurgical Devices (OVDs) used during the implantation of AIOLs can be removed from the natural lens capsule. The embodiment of the AIOL 100 illustrated in FIGS. 1A and 1B comprises three outer flow-through features 114. The outer flow-through features 114 can be detents, such as a recess, distributed circumferentially along the perimeter of the base 102. The flow-through features 114 can create passages between the outer perimeter of the AIOL 100 and an inner surface of an eye capsule (not shown) in which the AIOL 100 is implanted to allow fluid flow around an outer perimeter of the AIOL 100. In the illustrated embodiment, for example, the flow-through features 114 are formed in regions of the first component 106 and second component 108. Although three outer flow-through features 114 are illustrated, other embodiments may comprise fewer or more than illustrated. The outer flow-through features 114 may additionally provide rotational constraint to maintain the rotational orientation of the base 102 with respect to a patient's eye capsule when implanted.

The fixed lens 104 can include an optical or lens portion 116 and one or more tabs 118 (individually identified as a first tab 118a, a second tab 118b, and a third tab 118c in FIGS. 1A and 1B) extending radially outward from the lens portion 116. The lens portion 116 can have, define, and/or otherwise provide one or more fixed (e.g., unchanging, static, non-dynamically adjustable) optical characteristics. In at least some embodiments, for example, the lens portion 116 can have, define, and/or otherwise provide a fixed optical power, a fixed depth of field, and/or a fixed depth of focus. The lens portion 116 can include one or more of an asymmetrically powered lens (e.g., a toric lens), spherical lens, an aspheric lens, a plano-convex lens, a convex-concave lens, a convex-convex lens, or another suitable lens. In some embodiments, the lens portion 116 can have a diopter of between about 0 D and about 20 D, such as between about 8 D and about 20 D, between about 12.5 D and about 18.5 D, any diopter therebetween, or another suitable diopter. In other embodiments, the lens portion 116 can have a negative diopter, e.g., a diopter of between about −5 D and about −0.01 D, any diopter therebetween, or another suitable diopter.

At least a portion of one or more of the tabs 118 can be configured to be received by the base 102, for example, to couple or otherwise at least partially or fully prevent movement of the fixed lens 104 relative to the base 102. In the illustrated embodiment, for example, when the fixed lens 104 is positioned within the volume 110 (FIG. 1B), at least a portion of the first tab 118a is positioned within the first slot 112a, at least a portion of the second tab 118b is positioned within the second slot 112b, and at least a portion of the third tab 118c is positioned within the third slot 112c. The interaction between the tabs 118 and the slots 112 can at least partially or fully prevent unintended rotation of the fixed lens 104 relative to the base 102 and/or at least partially or fully prevent the fixed lens 104 from moving anteriorly and/or posteriorly relative to the base 102. In at least some embodiments, for example, the fixed lens 104 is expected to maintain a generally or substantially consistent position, orientation, rotational alignment, etc. relative to the base 102 unless or until one or both of the fixed lens 104 and the base 102 are manipulated by a practitioner or other user.

In some embodiments, individual ones of the tabs 118 can include one or more apertures or holes 120. Each of the apertures 120 can extend at least partially or fully through the corresponding tab 118, and can be engaged by a surgical tool to manipulate the fixed lens 104, such as during implantation and/or removal of the fixed lens 104. In some embodiments, the apertures 120 are arranged in pairs. In some embodiments, the apertures 120 are distributed in a circumferential pattern. In some embodiments, one or more of the apertures 120 have a different size (e.g., width or diameter) and/or shape than other apertures 120. For example, each of the apertures 120 may have a different size than each of the other apertures 120. Using apertures of varying size can allow for further visual confirmation of the rotational alignment of the fixed lens 104 (e.g., about the optical axis of the AIOL 100). Confirming the alignment/orientation of the fixed lens 104 is expected to reduce the risk that a toric lens or other non-annularly-symmetric fixed lens is improperly oriented with respect to the base 102 and/or native eye capsule into which the AIOL 100 is implanted. In some embodiments, the fixed lens 104 can include additional visual markers to indicate an orientation of the fixed lens 104 with respect to the base 102 and/or the native eye capsule.

FIG. 1C is a cross-section of the AIOL 100 taken along line 1C-1C of FIG. 1B. As best seen in FIG. 1C, the base 102 can define a first fluid chamber or reservoir 122 (which can also be referred to as an “outer fluid reservoir 122,” a “haptic reservoir 122,” and/or the like), and a second fluid chamber or reservoir 124 (which can also be referred to as an “inner fluid reservoir 124,” an “optical fluid chamber 124,” and/or the like), both of which can be filed with fluid (e.g., optical fluid, such as silicone oil or a solution comprising high molecular weight dextran, and/or including one or more fluids with an index of refraction greater than the aqueous humor of the eye). A channel 126 can extend between and fluidly couple the outer fluid reservoir 122 and the inner fluid reservoir 124 to allow the fluid to flow between the outer fluid reservoir 122 and the inner fluid reservoir 124. The outer fluid reservoir 122 can be defined at least partially between a first or outer wall 128 and a second or inner wall 130 of the first component 106 and/or a first or outer wall 132 and a second or inner wall 134 of the second component 108. The fluid contained within the outer fluid reservoir 122 and/or the inner fluid reservoir 124 can have a refractive index different than (e.g., less than or greater than) a refractive index of aqueous.

The outer fluid reservoir 122 can be configured to operate as a bellows to cause fluid flow between the outer fluid reservoir 122 and the inner fluid reservoir 124 in response to forces applied to the AIOL 100, for example, such that a change to a force applied to a portion of the AIOL 100 can cause fluid flow (via the channel 126) between the outer fluid reservoir 122 and the inner fluid reservoir 124. In at least some embodiments, for example, a radially compressive force applied to at least a portion of the AIOL 100, such as an outer perimeter of the base 102, can decrease a volume of the outer fluid reservoir 122 and/or increase a pressure within the outer fluid reservoir 122. This volume decrease and/or pressure increase can cause fluid within the outer fluid reservoir 122 to flow toward and/or into the inner fluid reservoir 124 and thereby expand the inner fluid reservoir 124, e.g., by increasing the volume and/or pressure within the inner fluid reservoir 124. When the radially compressive force is decreased or eliminated entirely, the volume of the outer fluid reservoir 122 can increase and/or the pressure within the outer fluid reservoir 122 can decrease and thereby cause fluid within the inner fluid reservoir 124 to flow toward and/or into the outer fluid reservoir 122. This fluid flow toward and/or into the outer fluid reservoir 122 can return the inner toward reservoir 124 toward and/or to an unexpanded state. Causing fluid flow from the outer fluid reservoir 122 toward and/or into the inner fluid reservoir 124 can transition the AIOL 100 from an at least general disaccommodated (e.g., negative accommodation) state toward and/or into a generally accommodated state. Likewise, causing fluid flow from the inner fluid reservoir 124 toward and/or into the outer fluid reservoir 122 can transition the AIOL 100 from an at least generally accommodated state toward and/or into a generally disaccommodated state.

The base 102 can include an adjustable lens 136 at least partially defined by the inner fluid reservoir 124, a first optical portion 138 of the first component 106, and a second optical portion 140 of the second component 108. The adjustable lens 136 can be positioned posterior to the fixed lens 104 and/or the lens portion 116 thereof. The adjustable lens 136 can have, define, and/or otherwise provide one or more ranges of dynamically adjustable optical characteristics. In at least some embodiments, for example, the adjustable lens 136 can have, define, and/or otherwise provide a range of dynamically adjustable optical powers, depths of focus, and/or depths of field. The first optical portion 138 and/or the second optical portion 140 can be planar members or optical membranes. In at least some embodiments, for example, one or both of the first optical portion 138 and the second optical portion 140 can be optical membranes integrally formed with the other portions of the first component 106 and the second component 108, respectively. In other embodiments, one or both of the first optical portion 138 and the second optical portion 140 can include one or more deformable lenses that are configured to provide one or more fixed optical characteristics, and flex or deflect based at least partially in response to changes to a volume and/or fluid pressure within the inner fluid reservoir 124. In at least some embodiments, for example, fluid flow between the outer fluid reservoir 122 and the inner fluid reservoir 124 can cause one or both of the first component 106 and the second component 108 to bend or flex in response to an increase or decrease in a volume of fluid and/or a fluid pressure within the inner fluid reservoir 124. This increase or decrease in the fluid volume and/or pressure within the inner fluid reservoir 124 can change (e.g., increase or decrease) one or more of the optical characteristics of the adjustable lens 136 within the associated range of dynamic adjustability provided by the adjustable lens 136. That is, the adjustable lens 136 can have one or more optical characteristics at a given time, each of those optical characteristics can be one value within a range of values, and increasing or decreasing the fluid volume/pressure within the inner fluid reservoir 124 can dynamically adjust the value of one or more of the optical characteristics to another value within the range of values for that optical characteristic. In at least some embodiments, for example, the adjustable lens 136 provides a range of optical powers, and increasing or decreasing the fluid volume/pressure within the inner fluid reservoir 124 can dynamically adjust the optical power of the adjustable lens 136 within the range of optical powers. Additionally, or alternatively, the adjustable lens 136 provides one or more depths of focus within a range of depths of focus, and increasing or decreasing the fluid volume/pressure within the inner fluid reservoir 124 can dynamically adjust the depth of focus of the adjustable lens 136 within the range of depths of focus. In these and/or other embodiments, the adjustable lens 136 provides one or more depths of field within a range of depths of field, and increasing or decreasing the fluid volume/pressure within the inner fluid reservoir 124 can dynamically adjust the depth of field of the adjustable lens 136 within the range of depths of field. In one particular example, the adjustable lens 136 is dynamically adjustable between a first optical power and a second optical power. The adjustable lens 136 can have the first optical power when the AIOL 100 is in the disaccommodated state and transitioning the AIOL 100 to the accommodated state can dynamically adjust the optical power provided by the adjustable lens 136 to the second optical power. More generally, in the accommodated or disaccommodated state, the adjustable lens 136 can have a diopter of between about 5 D and about 7.5 D, such as between about 5.8 D and about 7.3D, any diopter therebetween, or another suitable diopter. Additionally, or alternatively, the diopter of the adjustable lens 136 can have a range of adjustment of up to 1.5D, 3D, 4D, 5D, 7.5D, any range therebetween, or another suitable range.

FIGS. 1D and 1E are perspective views of the AIOL 100 of FIGS. 1A-1C with the fixed lens 104 engaged with the base 102. In some embodiments, one or both of the base 102 and the fixed lens 104 can be configured to rotate relative to one another. In the illustrated embodiment, for example, the slots 112 of the base 102 can be configured to slidably receive the tabs 118 of the fixed lens 104, such that the fixed lens 104 can be rotated within the volume 110 in a first or counter-clockwise direction R1 (FIG. 1D) and/or a second or clockwise direction R2 (FIG. 1E) relative to the base 102 about an optical axis O of the AIOL 100. In some embodiments, one or more of the apertures 120 in the tabs 118 can be used to rotate the fixed lens 104 relative to the base 102.

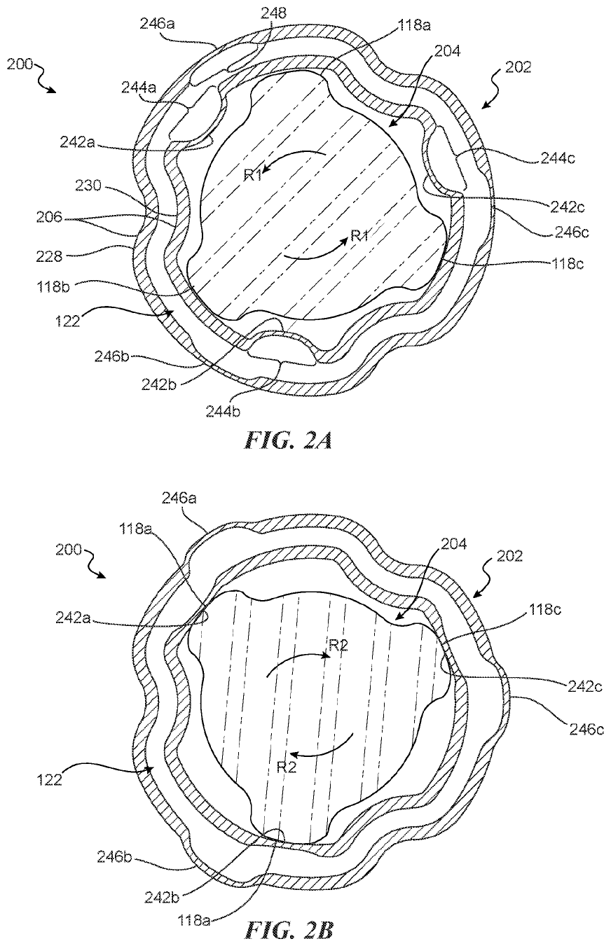

FIGS. 2A and 2B are top cross-sections of an adjustable intraocular lens 200 (“AIOL 200”) configured in accordance with embodiments of the present technology. At least some aspects of the AIOL 200 can at least generally similar or identical in structure and/or function to one or more features of the AIOL 100 of FIGS. 1A-1E, with like reference numbers used to identify at least generally similar or identical features. In the illustrated embodiment, for example, the AIOL 200 includes a base 202 that can be at least generally similar in structure and/or function to the base 102 of FIGS. 1A-1E and a fixed lens 204 that can be at least generally similar in structure to the fixed lens 104 of the FIGS. 1A-1E. For example, although not shown in FIGS. 2A and 2B merely for purposes of clarity, the base 202 can define an adjustable, fluid-filled lens that is at least generally similar in structure and/or function to the adjustable lens 136 of FIG. 1C. The AIOL 200 is further configured such that rotating the fixed lens 204 relative to the base 202 can change or shift the range of dynamic adjustability of one or more optical characteristics of the base 202. For example, as described further below, rotating the fixed lens 204 can increase or decrease a volume of fluid within the adjustable lens defined by the base 202 and thereby increase or decrease the optical power provided by the adjustable lens.

Referring to FIG. 2A, an inner wall 230 of the base 202 can include one or more actuator portions 242 (individually identified as a first actuator portion 242a, a second actuator portion 242b, and a third actuator portion 242c in FIGS. 2A and 2B). Although the illustrated embodiment includes three actuator portions 242, other embodiments can include more or fewer actuator portions 242, such as at least one, two, four, five, or another suitable number of actuator portions 242. In at least some embodiments, the number of actuator portions 242 can be less than, equal to, or greater than the number of tabs 118. Each of the actuator portions 242 be defined at least partially by a thinned region 244 (individually identified as a first thinned region 244a, a second thinned region 244b, and a third thinned region 244c in FIGS. 2A and 2B) of the inner wall 230. The inner wall 230 can be configured such that rotation of the fixed lens 204 causes deflection of the actuator portions 242 without or substantially without deflection of one or more other portions of the inner wall 230.

Rotating the fixed lens 204 relative to the base 202 can cause a change in the contact and/or the physical interaction between the fixed lens 204 and the base 202 that causes fluid flow between the outer fluid reservoir 122 and the inner fluid reservoir 124. The fluid flow between the outer fluid reservoir 122 and the inner fluid reservoir 124 can thereby adjust one or more of the optical characteristics of the adjustable lens of the base 202. In at least some embodiment, for example, rotating the fixed lens 204 can move one or more of the tabs 118a-b into contact with a corresponding one of the actuator portions 242a-c. For example, as illustrated in FIGS. 2A and 2B together, rotating the fixed lens 204 in the first direction R1 can cause the first tab 118a to contact the first actuator portion 242a, the second tab 118b to contact the second actuator portion 242b, and/or the third tab 118c to contact the third actuator portion 242c. In some embodiments, the fixed lens 204 is configured to at least partially resist rotation relative to the base 202 and/or the base 202 is configured to at least partially resist rotation of the fixed lens 204 relative to the base 202, and rotating the fixed lens 204 includes rotating the fixed lens 204 with sufficient force to overcome this resistance. For example, in at least some embodiments, the fixed lens 204 and/or the base 202 include friction contact surfaces, keyed or toothed surfaces to provide mechanical interference, and/or one or more other features configured to at least partially resist rotation of the fixed lens 204 relative to the base 202.

As the fixed lens 204 is rotated in the first direction R1, the tabs 118 can contact the corresponding actuator portions 242 at a same time, or at least substantially simultaneously, or one or more of the tabs 118 can contact the corresponding actuator portion 242 in series or sequence, e.g., after at least one of the tabs 118 is contacting the corresponding actuator portion 242 and/or before at least one of the tabs 118 contacts the corresponding actuator portion 242. Each of the tabs 118 can drive/deflect the corresponding actuator portion 242 radially outward, into the outer fluid reservoir 122, and/or toward an outer wall 228 of the base 202. The radially outward deflection of the actuator portions 242 into the outer fluid reservoir 122 can decrease a volume of the outer fluid reservoir 122 and/or increase a pressure within the outer fluid reservoir 122 to thereby drive fluid from the outer fluid reservoir 122 toward and/or into the inner fluid reservoir 124 (FIG. 1C) and, accordingly, increase an optical power of the adjustable lens by, for example, changing a geometry of the adjustable lens. In some embodiments, increasing a volume of fluid within the inner fluid reservoir 124 can cause the first optical portions 138 (FIG. 1C) to deform anteriorly and/or the second optical portion 140 (FIG. 1C) to deform posteriorly, which can in turn increase a curvature of the adjustable lens and thereby increase the optical power provided by the adjustable lens. Because the adjustable lens has a range of dynamically adjustable optical characteristics, changing the geometry of the adjustable lens can change or shift the range of dynamic adjustability of the adjustable lens. This, in turn, can change how the adjustable lens responds to accommodative/disaccommodative forces from the patient's eye. For example, rotating the fixed lens 204 as described above can not only increase the optical power of the adjustable lens when the AIOL 200 is an at least generally disaccommodated state, it can also shift the range of optical powers to increase the maximum optical power to which the adjustable lens can be adjust when the AIOL 200 is an at least generally or maximally accommodated state. Put differently, the adjustable lens can have a first range of dynamically adjustable optical powers including a first minimum optical power and a first maximum optical power before the fixed lens 204 is rotated and, after rotating the fixed lens 204, the adjustable lens can have a second (e.g., higher) range of dynamically adjustable optical powers including a second minimum optical power and second maximum optical power. The second minimum optical power can be greater than the first minimum optical power and/or the second maximum optical power can be greater than the first maximum optical power. The same can be true for other optical characteristics of the adjustable lens, such as depth of focus and/or depth of field. That is, the adjustable lens can have a first range of dynamically adjustable depths of focus before rotating the fixed lens 204 and, after rotating the fixed lens 204, the adjustable lens can have a second range of dynamically adjustable depths of focus with greater minimum and/or maximum depths of focus than the first range. Likewise, the adjustable lens can have a first range of dynamically adjustable depths of field before rotating the fixed lens 204 and, after rotating the fixed lens 204, the adjustable lens can have a second range of dynamically adjustable depths of field with greater minimum and/or maximum depths of field than the first range.

Rotating the fixed lens 204 in the second direction R2, opposite the first direction R1, can allow the actuator portions 242 to return to their undeflected state. For example, rotating the fixed lens in the second direction R2 can move the tabs 118 out of contact with the actuator portions 242 and allow the actuators portions 242 to bend or flex inwardly, e.g., toward and/or to the undeflected state, such as shown in FIG. 2A. Such movement of the actuator portions 242 can increase the volume of the outer fluid reservoir 122 and/or decrease the pressure within the outer fluid reservoir 122, and, accordingly, allow or cause fluid within the inner fluid reservoir 124 (FIG. 1C) to flow toward and/or into the outer fluid reservoir 122 and thereby decrease an optical power of the adjustable lens. Because the second direction R2 is opposite the first direction R1, rotating the fixed lens 204 in the second direction R2 can have the opposite effect on one or more of the ranges of dynamically adjustable optical characteristics of the adjustable lens. That is, rotating the fixed lens 204 in the second direction R2 can return the range of dynamically adjustable optical powers, depth(s) of focus, and/or depth(s) of field from a second (e.g., higher) range to a first (e.g., lower) range with minimum and/or maximum values less than those in the second range.

In some embodiments, the actuator portions 242 can apply a force to the corresponding tabs 118 (e.g., equal and opposite to the force applied to the actuator portions 242 by the tabs 118) that resists, inhibits, or entirely prevents, rotation of the fixed lens 204 relative to the base 202. Additionally, or alternatively, one or more of the actuator portions 242 can include friction contact surfaces, keyed or toothed surfaces to provide mechanical interference, and/or one or more other features configured to at least partially resist rotation of the fixed lens 204 relative to the base 202. Accordingly, in these and/or other embodiments, rotating the fixed lens 204 relative to the base 202 can include rotating the fixed lens 204 with sufficient force to overcome any resistance to that rotation.

In some embodiments, the outer wall 228 of the base 202 can include one or more relief portions 246 (individually identified as a first relief portion 246a, a second relief portion 246b, and a third relief portion 246c in FIGS. 2A and 2B). Each of the relief portions 246 can be defined at least partially by a thinned region 248 (one labeled in FIG. 2A) of the outer wall 228. The relief portions 246 can be configured to bend or flex (e.g., radially outwardly or inwardly) after and/or in response to rotation of the fixed lens 204. For example, when rotating the fixed lens 204 in the first direction RI (FIG. 2A), the corresponding decrease in the volume of outer fluid reservoir 122, and/or the increase of the pressure within the outer fluid reservoir 122, can bend or flex one or more of the relief portions 246 outwardly, away from the inner wall 230, and thereby increase the volume of the outer fluid reservoir 122and/or decrease the pressure within the outer fluid reservoir 122. In at least some embodiments, relief portions 246 are configured to reduce the tension, strain, and/or one or more other forces on the base 202 generated in response to rotating the fixed lens 204 while still allowing the rotation of the fixed lens 204 to change the optical power provided by the base 202. For example, the volume increase/pressure decrease associated with the radially outward deflection of the relief portions 246 can be less than the volume decrease/pressure increase associated with the radially outward deflection of the actuator portions 242, such that rotating the fixed lens 204 can cause a net volume decrease/pressure increase within the outer fluid reservoir 122 and a net fluid flow from the outer fluid reservoir 122 toward and/or into the inner fluid reservoir 124 (FIG. 1C). The outer wall 228 can be configured such that rotating the fixed lens 204 causes the relief portions 246 to bend or flex without or substantially without bending or flexing one or more other portions of the outer wall 228. As noted above, the relief portions 246 are expected to reduce or prevent mechanical stresses or wear associated with rotation of the fixed lens 204 and the corresponding changes to the volume/pressure of the outer fluid reservoir 122, which can increase the operational lifetime of the AIOL 200. Additionally, or alternatively, the relief portions 246 are expected to increase the sensitivity of the AIOL 200 to external accommodating forces, such as compressive forces applied by a capsular bag of a patient's eye, when the relief portions 246 are in the radially outwardly deflected state, such as shown in FIG. 2B. In at least some embodiments, for example, the radially outward deflection of the relief portions 246 can increase the effective diameter of the base 202 and/or improve the accommodative response of the adjustable lens (e.g., the adjustable lens 136 of FIG. 1C) when external accommodative forces are applied to the AIOL 200.

One or more aspects of the fixed lens 204 can be configured based at least in part on the interaction between the base 202 and at least a portion of the patient's eye (e.g., a capsular bag of the patient's eye). In at least some embodiments, for example, the base 202 can be positioned within the capsular bag and changes in one or more optical characteristics of the base due at least in part to the elastic deformation/accommodation of the base 202 can be used to select an appropriately configured fixed lens 204, e.g., a fixed lens 204 with one or more fixed optical characteristics appropriate for the patient based on an observed accommodative response of the base 202 after implantation. Additionally or alternatively, the observed accommodative response of the base 202 can be used to select a fixed lens 204 based at least partially on a predicted/expected accommodative response of the AIOL 200 after the fixed lens 204 has been rotated relative to the base 202. As shown in FIG. 2B, rotating the fixed lens 204 to outwardly-flex the relief portions 246 can increase an outer diameter of the base 202. In some embodiments, this increase to the outer diameter of the base 202 is expected to improve the fit between the AIOL 200 and the patient's capsular bag. For example, if the base 202 is undersized relative to the capsular bag, a user can rotate the fixed lens 204 in the first direction R1 to redistribute fluid within the base 202 and cause the relief portions 246 to engage the capsular bag of the patient's eye. In other embodiments, the AIOL 200 can be implanted in the configuration shown in FIG. 2B and, if the base 202 is oversized relative to the capsular bag, a user can rotate the fixed lens 204 in the second direction R2 to redistribute fluid within the base 202 to reduce the outer diameter of the base 202 and improve the fit of the base 202 within the capsular bag. Whether the base 202 is undersized or oversized are two examples of sizing or fit characteristics associated with the implanting the base 202 within the capsular bag. Based on a difference between one or more of the fit characteristics (e.g., an outer diameter of the base 202) and an associate threshold value (e.g., an inner diameter of a disaccommodated or accommodated capsular bag), the fixed lens 204 can be rotated to improve the fit of the base 202. Additional fit characteristics that can be used to determine whether a change to the fixed lens 204 would improve the performance of the base 202 include a value (e.g., a minimum and/or maximum value) and/or range of values for one or more of the optical characteristics provided by the adjustable lens. Accordingly, a user can rotate the fixed lens 204 to improve, or optimize, one or more of the optical characteristics of the adjustable lens. In at least some embodiments, for example, adjusting the fixed lens 204 can improve the accommodative response of the adjustable lens. If the base 202 is oversized relative to the capsular bag, the adjustable lens can be incapable of achieving one or more optical characteristics associated with a fully disaccommodated state of the capsular bag(e.g., far vision). When oversized, the periphery of the base 202 (including, e.g., the outer fluid reservoir 122) can press against the capsular bag and lead to more fluid than expected being redistributed toward and/or into the inner fluid reservoir. This means that, during periods of disaccommodation, one or more of the optical properties of the adjustable lens may be higher than expected and, accordingly, the patient's ability to focus on distant objects can be negatively impacted. However, as described above, rotating the fixed lens 204 (e.g., in the second direction R2) to redistribute fluid from the inner fluid reservoir toward and/or into the outer fluid reservoir 122 can increase the volume/pressure of fluid within the outer fluid reservoir 122, decrease the volume/pressure of fluid within the inner fluid reservoir and, accordingly, improve the patient's ability to focus on distant objects. The same can be true for the patient's ability to focus on near objects when the base 202 is undersized. During periods of accommodation, one or more of the optical properties of an adjustable lens in an undersized base may be lower than expected and, accordingly, the patient's ability to focus on near objects can be negatively impacted. However, as described above, rotating the fixed lens 204 (e.g., in the first direction R1) to redistribute fluid from the outer fluid reservoir 122 toward and/or into the inner fluid reservoir can increase the volume/pressure of fluid within the inner fluid reservoir and, accordingly, improve the patient's ability to focus on near objects.

Although a first component 206 of the base 202 is illustrated as including the actuator portions 242 and the relief portions 246 in FIGS. 2A and 2B, in these and/or other embodiments a second component of the base 202, which can be at least generally similar in structure and/or function to the second component 108 of FIGS. 1A-1C, can include the actuator portions 242 and/or the relief portions 246. In at least some embodiments, for example, an inner wall of the second component (e.g., the inner wall 134 of FIG. 1C) can include one or more of the actuator portions 242 and/or an outer wall of the second component (e.g., the outer wall 132 of FIG. 1C) can include one or more of the relief portions 246.

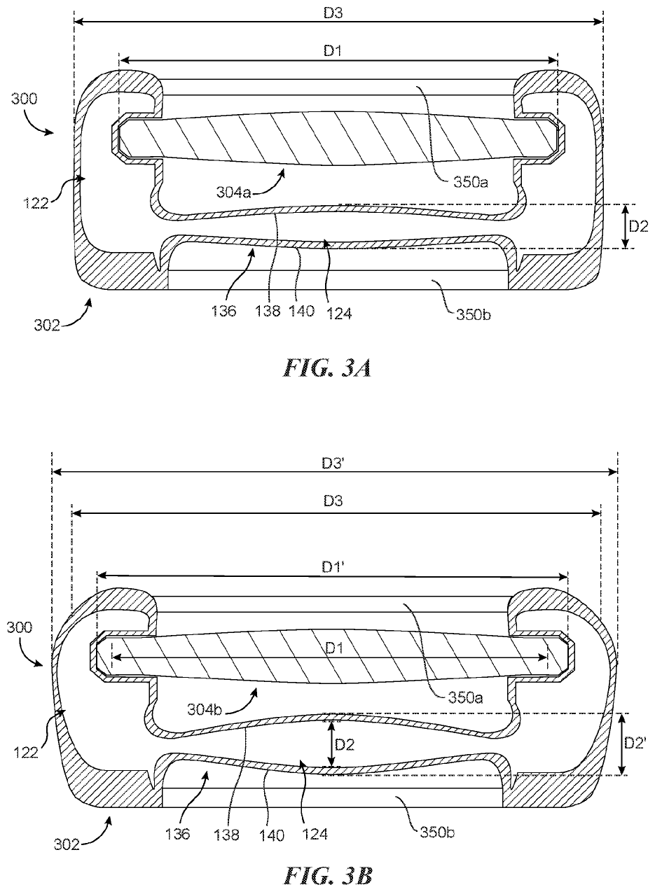

FIGS. 3A and 3B are side cross-sections of an adjustable intraocular lens 300 (“AIOL 300”) configured in accordance with embodiments of the present technology. The AIOL 300 can include at least some aspects that are at least generally similar or identical in structure and/or function to the AIOL 100 of FIGS. 1A-1E and/or the AIOL 200 of FIGS. 2A and 2B, with like reference numbers used to identify at least generally similar or identical features. In the illustrated embodiment, for example, the AIOL 300 includes a base 302 that is at least generally similar to the base 102 of FIGS. 1A-1E and/or the base 202 of FIGS. 2A and 2B. However, the base 302 is further configured to receive a plurality of differently-dimensioned fixed lenses. Referring to FIG. 3A, a first fixed lens 304a having a first fixed lens dimension DI (e.g., a diameter, width, length, etc.) is received by the base 302, and the adjustable lens 136 has a first adjustable lens dimension D2 (e.g., a height, center thickness, etc.).

Referring to FIG. 3B, a second fixed lens 304b having a second fixed lens dimension D1′ greater than the first fixed lens dimension DI is received by the base 302, and the adjustable lens 136 has a second adjustable lens dimension D2′ greater than the first adjustable lens dimension D2. In FIG. 3B, the first dimensions D1, D2 described previously with reference to the first fixed lens 304a are reproduced for the sake of comparison with the dimensions associated with the second fixed lens 304b. As best seen in FIG. 3B, changing the size of the fixed lens can change the contact and/or physical interaction between the fixed lens and the base 302 which, in turn, can redistribute fluid between the outer fluid reservoir 122 and the inner fluid reservoir 124 and thereby cause a corresponding adjustment to the optical power and/or other optical characteristics of the adjustable lens 136. In the illustrated embodiment, for example, the increase to the second fixed lens dimension D1′ of the second fixed lens 304b relative to the first fixed lens dimension D1 of the first fixed lens 304a can cause a decrease in the volume of the outer fluid reservoir 122 and/or an increase to the pressure within the fluid reservoir 122 and thereby cause fluid within the outer fluid reservoir 122 to flow toward and/or into the inner fluid reservoir 124. The adjustable lens 136 can swell to accommodate this fluid, increasing from the first adjustable lens dimension D2 to the second adjustable lens dimension D2′ of the adjustable lens 136 and thereby adjusting (e.g., increasing) the optical power and/or other optical characteristics provided by the adjustable lens 136.

In some embodiments, the base 302 can bend or flex to accommodate the increased size of the second fixed lens 304b (FIG. 3B). For example, the base 302 has a first base dimension D3 (e.g., a diameter, width, length, etc.) when the base 302 receives the first fixed lens 304a (FIG. 3A) and a second base dimension D3′ greater than the first base dimension D3 when the base lens receives the second fixed lens 304b (FIG. 3B). In some embodiments, the increased base dimension D3′ can cause the volume of the outer fluid reservoir 122 to increase and/or the pressure within the outer fluid reservoir 122 to decrease. However, the volume increase/pressure decrease associated with the increased base dimension D3′ can be less than the volume decrease/pressure increase associated with the increased fixed lens dimension D1′ of the second fixed lens 304b, such that the second fixed lens 304b can cause a net decrease to the volume of the outer fluid reservoir 122 and/or a net increase to the pressure within the outer fluid reservoir 122 and thereby cause fluid flow toward and/or into the inner fluid reservoir 124 as described above.

In some embodiments, the change in dimension of the base 302 is expected to reduce or prevent mechanical stresses or wear associated with larger fixed lenses (e.g., the fixed lens 304b) and the corresponding changes to the volume/pressure of the outer fluid reservoir 122, which can increase the operational lifetime of the AIOL 300. Additionally, or alternatively, the increased base dimension D3′ of the base 302 is expected to increase sensitivity of the AIOL 300 to external accommodating forces, such as compressive forces applied by a capsular bag of a patient's eye. In at least some embodiments, the increased sensitivity to the external accommodative forces can improve the accommodative response of the adjustable lens 136 when the external accommodative forces are applied to the AIOL 300. In these and other embodiments, the base 302 can include one or more support structures or components 350 (individually identified as a first or anterior support component 350a and a second or posterior support component 350b in FIGS. 3A and 3B) configured to at least partially or fully prevent deformation of the base 302, for example, in an anterior-to-posterior direction. In some embodiments, the support components 350 can be configured to oppose, or at least partially oppose, forces (e.g., compressive forces) from the patient's eye and thereby allow these forces to redistribute fluid within the base 302 to change (i) a base value of one or more optical characteristics of the adjustable lens 136 and/or (ii) a minimum and/or maximum value of a range of adjustability of one or more of the optical characteristics of the adjustable lens 136, as described previously herein.

In some embodiments, one or more aspects of the fixed lenses 304 can be configured based at least in part on the interaction between the base 302 and at least a portion of the patient's eye (e.g., a capsular bag of the patient's eye). In at least some embodiments, for example, the base 302 can be positioned within the capsular bag and the elastic deformation/accommodation of the base 302 and/or other changes to optical characteristics of the base 302 in response to the interaction between the base 302 and the capsular bag can be used to select appropriately configured fixed lenses 304b, as described previously with reference to FIGS. 2A and 2B. Additionally or alternatively, the observed accommodative response of the base 302 can be used to select an appropriately sized fixed lens 304 based at least partially on a predicted/expected accommodative response of the AIOL 300 after the first fixed lens 304a has been replaced with the second fixed lens 304b and/or after the second fixed lens 304b has been replaced with the first fixed lens 304a.

In some embodiments, the choice between the first fixed lens 304a and the second fixed lens 304b can be based, at least in part, on the size of the base 302 relative to the capsular bag of the patient's eye. If the second base dimension D3′ is oversized relative to the capsular bag, the second fixed lens 304b can be replaced with the first fixed lens 304a to reduce the base dimension to the first base dimension D3 and improve the fit of the base 302 within the capsular bag. Likewise, if the first base dimension D3 is undersized relative to the capsular bag, the first fixed lens 304a can be replaced with the second fixed lens 304b to increase the base dimension to the second base dimension D3′ and improve the fit of the base 302 within the capsular bag. As noted above, whether the base 302 is undersized or oversized are two examples of sizing or fit characteristics associated with the implanting the base 302 within the capsular bag. Based on a difference between one or more of the fit characteristics (e.g., an outer diameter of the base 302) and an associate threshold value (e.g., an inner diameter of a disaccommodated or accommodated capsular bag), the fixed lens 304 can be replaced to improve the fit of the base 302. Additional fit characteristics that can be used to determine whether replacing the fixed lens 304 would improve the performance of the base 302 include a value (e.g., a minimum and/or maximum value) and/or range of values for one or more of the optical characteristics provided by the adjustable lens 124. Accordingly, a user can replace the fixed lens 304 to improve, or optimize, one or more of the optical characteristics of the adjustable lens 124. In at least some embodiments, example, the choice between the first fixed lens 304a and the second fixed lens 304b can be based, at least in part, on improving the accommodative response of the adjustable lens 136. If the base 302 is oversized relative to the capsular bag, the adjustable lens 136 can be incapable of achieving one or more optical characteristics associated with a fully disaccommodated state of the capsular bag (e.g., far vision). When oversized, the periphery of the base 302 (including, e.g., the outer fluid reservoir 122) can press against the capsular bag and lead to more fluid than expected being redistributed toward and/or into the inner fluid reservoir 124. This means that, during periods of disaccommodation, one or more of the optical properties of the adjustable lens 136 may be higher than expected and, accordingly, the patient's ability to focus on distant objects can be negatively impacted. However, as described above, replacing the second fixed lens 304b with the first (e.g., smaller) fixed lens 304a can redistribute fluid from the inner fluid reservoir 124 toward and/or into the outer fluid reservoir 122, decrease the volume/pressure of fluid within the inner fluid reservoir 124 and, accordingly, improve the patient's ability to focus on distant objects. The same can be true for the patient's ability to focus on near objects when the base 302 is undersized. During periods of accommodation, one or more of the optical properties of the adjustable lens 136 in an undersized base may be lower than expected and, accordingly, the patient's ability to focus on near objects can be negatively impacted. However, as described above, replacing the first fixed lens 304a with the second (e.g., larger) fixed lens 304b to redistribute fluid from the outer fluid reservoir 122 toward and/or into the inner fluid reservoir 124 can increase the volume/pressure of fluid within the inner fluid reservoir 124 and, accordingly, improve the patient's ability to focus on near objects.

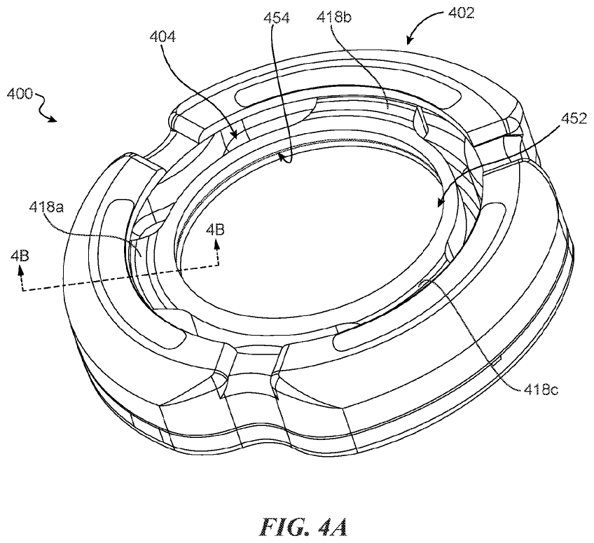

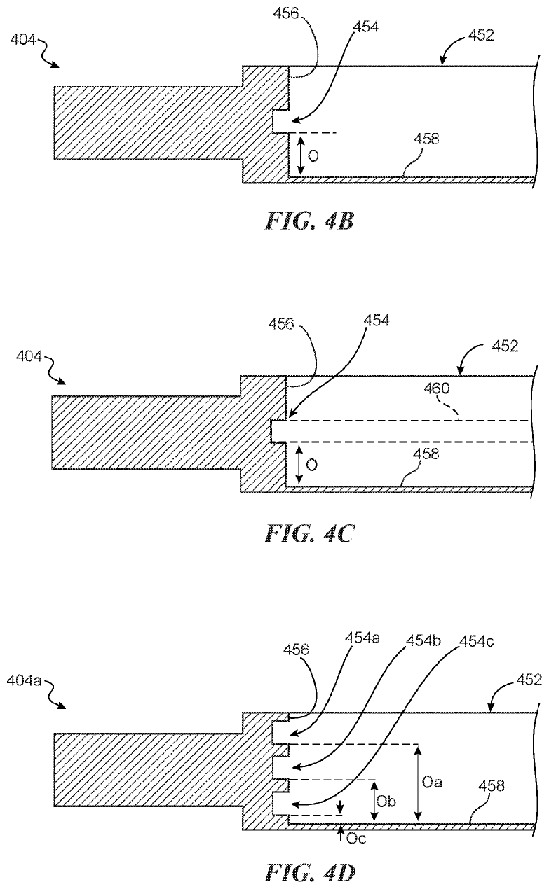

FIG. 4A illustrates an AIOL 400 configured in accordance with embodiments of the present application. The AIOL 400 includes a base 402 and an adapter or cartridge 404. The base 402 can be at least generally similar or identical in structure and/or function to the base 102 of FIGS. 1A-1E, the base 202 of FIGS. 2A and 2B, and/or the base 302 of FIGS. 3A and 3B. Additionally, or alternatively, the cartridge 404 can be configured to matingly engage the base 402, and/or adjust one or more optical properties thereof, in a manner that is at least generally similar or identical to the fixed lens 104 of FIGS. 1A-1E, the fixed lens 204 of FIGS. 2A and 2B, and/or one or more of the fixed lenses 304 of FIGS. 3A-3B. In some embodiments, the cartridge 404 can be rotated relative to the base 402 and/or replaced with a cartridge of another size to change (i) one or more of the optical properties of an adjustable lens of the base 402 and/or (ii) a minimum and/or maximum value of a range of adjustability of one or more of the optical characteristics of the adjustable lens. Additionally, the cartridge 404 can be annular (e.g., ring-shaped) and define a cavity 452 (e.g., a central cavity) having interior portions configured to receive a standard lens (e.g., an off-the-shelf or other commercially available lens), such as a lens having one or more fixed optical characteristics. In the illustrated embodiment, for example, the cartridge structure 404 includes one or more lens-receiving or support features 454 configured to receive at least a portion (e.g., the haptics) of such a standard lens. the cartridge structure 404 can include a single lens-receiving feature 454 that extends around/about the entire cavity 452, or multiple lens-receiving features 454 that can each extend around all or a portion of the cavity 452. One such example is described in greater detail below with reference to FIG. 4D.

In some embodiments, the cartridge structure 404 has, defines, and/or otherwise provides one or more optical characteristics, in addition to the one or more optical characteristics provided by a standard lens (not shown) received within the cavity 452. In other embodiments, the cartridge structure 404 itself does not or substantially does not have any optical characteristics and/or is not otherwise configured to provide an optical correction.

The cartridge structure 404 may include tabs 418a-b configured to allow the cartridge structure 404 to be releasably fixed within the base 402, as shown in FIG. 4A. These tabs 418 can beneficially allow easy exchange of the cartridge structure 404. It is expected that coupling the standard lens directly to the base 402 may alter the optical characteristics (e.g., optical power, depth of focus, depth of field, and/or one or more ranges of adjustability thereof) of the base 402. Accordingly, the cartridge structure 404 can be configured to interface with the base 402, as described previously herein, while also holding the standard lens without or substantially without altering the expected optical characteristics of the base 402.

FIG. 4B is a side cross-sectional view of the cartridge structure 404 taken along section line 4B-4B of FIG. 4A. The lens receiving feature 454 can be formed in a sidewall 456 defining the cavity 452. In the illustrated embodiment, the lens receiving feature 454 includes a slot or recessed area in the sidewall 456. In other embodiments, the lens receiving feature 454 can include one or more protrusions, or have one or more suitable structures configured to releasably receive one or more standard lenses. The lens receiving features 454 can spaced apart from a base or posterior surface 458 of the cavity 452 and/or the cartridge structure 404 by an offset. The offset O can be a distance between about 0.01 mm and about 1 mm, such as at least 0.1 mm, 0.2 mm, 0.5 mm, 0.7 mm, any distance therebetween, or another suitable distance. In some embodiments, the base 458 can be a posterior-most surface of the cartridge structure 404. Although the base 458 of the cavity 452 is illustrated as being planar or substantially planar in the embodiment illustrated in FIG. 4B, in other embodiments the base 458 of the cavity 452 can be convex, concave, curved, or have another suitable shape. In these and other embodiments, the base 458, and/or one or more other suitable portions of the cartridge 404, can have one or more optical characteristics and/or be otherwise configured to provide an optical correction, as described previously with reference to FIG. 4A. In further embodiments, the base 458 can be omitted such that the cavity 452 extends fully-through the cartridge structure 404 in a direction at least generally parallel to the sidewall 456.

FIG. 4C is the side cross-sectional view of the cartridge structure 404 of FIG. 4B and a standard lens 460 (shown in dashed line) in accordance with embodiments of the present technology. The standard lens 460 can be a non-accommodating lens and/or a lens having one or more fixed optical characteristics, including an off-the-shelf lens or other commercially-available lens. As illustrated in FIG. 4C, at least a portion of the standard lens 460 can be positioned or docked within the lens-receiving feature 454, such that the lens-receiving features 454 can support the standard lens 460 at the offset O from the base 458 of the cavity 452.

FIG. 4D is a side cross-sectional view of another cartridge structure 404 a configured in accordance with embodiments of the present technology. The cartridge structure 404a can include at least some features that are at least generally similar or identical in structure and/or function to the cartridge structure 404 of FIGS. 4B and 4C. Additionally, the cartridge structure 404a includes a plurality of lens-receiving features 454a-c (individually identified as a first lens-receiving features 454a, a second lens-receiving feature 454b, and a third lens-receiving feature 454c). Each of the lens-receiving features 454a-c can be formed in the same sidewall 456 and at respective offsets Oa-c (individually identified as a first offset Oa for the first lens-receiving feature 454a, a second offset Ob for the second lens-receiving feature 454b, and a third offset Oc for the third lens-receiving feature 454c) from the base 458. The lens-receiving features 454a-c can be arranged in an anterior-to-posterior alignment, such as with the third lens-receiving feature 454c being positioned anterior to the base 458 of the cavity 452, the second lens-receiving feature 454b positioned anterior to the third lens-receiving feature 454c, and the first lens-receiving features 454a positioned anterior to the second lens-receiving feature 454b, as shown in FIG. 4D. In these and other embodiments, the second offset Ob can be greater than the third offset, and the first offset Oa can be greater than the second offset Ob.

The cartridge structure 404a, with multiple lens-receiving features 454a-c having different offsets, allows the offset between a standard lens and the base 458 to be selectively adjusted, e.g., by a practitioner or other user, such as to improve or adjust the optical correction provided by the cartridge structure 404a. Additionally, or alternatively, multiple standard lens can be docked with the cartridge structure 404. For example, all or a subset (e.g., at least one, at least two, at least three, etc.) of the lens-receiving features 454 of the cartridge structure 404a can receive a standard lens, such as shown and described with reference to FIG. 4C. Although the cartridge structure 404a includes three lens-receiving features 454a-c in the embodiment illustrated in FIG. 4D, in other embodiments the cartridge structure 404a can include more or fewer lens-receiving features, such as at least two, four, five, six, or another suitable number of lens-receiving features.

The AIOL devices described herein may be implanted by preparing the eye and removing the native lens from the capsule in any appropriate manner. The fluid-filled structure may then be placed in the capsule of the eye. The patient may then be evaluated for one or more base optical characteristics and/or astigmatic correction, and a fixed lens is selected to provide the desired based optical characteristics or astigmatic correction for the fluid-filled structure in the implanted state in the capsule of the eye. The specific fixed lens is then inserted into the previously implanted fluid-filled structure of the AIOL. The chosen fixed lens may then be coupled to the fluid-filled structure within the eye capsule. This is possible in the AIOLs of the present technology because the fixed lenses are attached to the anterior first component of the AIOLs. As described above, one or more of the fluid-filled accommodating structure or fixed lens may each be flexible such that they may be reconfigured (e.g., folded) to a reduced-profile delivery configuration for delivery into the lens capsule. In some instances, it may be required to make a further correction to the fixed portion after the time of the surgery. Such instance may occur anywhere from days to years after the surgery. At such times, the patient may return to the physician and the fixed lens may be replaced with a new fixed lens having a different optical power or other prescription. In such instances, the new prescription may be characterized prior to or after removal of the original fixed lens. In some instances, the new fixed lens may be fabricated and implanted at the time of the examination, in others the patient may return for implantation of the fixed lens sometime after the examination.

Several embodiments of the present technology are directed to a kit having an accommodating structure and a first fixed lens that has no optical base power or other optical characteristics. The kit can further include one or more second fixed lenses having various based powers or other optical characteristics. In practice, the accommodating structure can be implanted into the native eye capsule, and then the first fixed lens can be coupled to the accommodating structure. The optical properties of the implanted accommodating structure can then be assessed in situ with the first fixed lens in place to determine the desired optical characteristics of the fixed lens. If the optical characteristics of the assembled accommodating structure and first fixed lens without a base power are appropriate, then the system can remain implanted without additional changes. However, if a different base power or some other optical property is desired (e.g., toric or other asymmetrical optics), then the first fixed lens without a base power can be replaced with a second fixed lens having the desired optical characteristics based on the optical characteristics of the implanted accommodating portion with a fixed lens attached.

In some embodiments, the fixed portion of the AIOL may be fabricated from materials different from the accommodating portion. Such materials include hydrophilic or hydrophobic methacrylate or silicones and any other materials traditionally used in non-accommodating IOLs. The fixed lens may be fabricated from materials harder than those used for the accommodating portion. One or both of the accommodating portion/lens and the fixed portion/lens may be machined, cast molded (e.g., reactive cast molded), injected molded, and/or formed by other processes or combinations of processes. Any or all of the structures described herein may be constructed from a transparent or translucent material. For example, the above-described accommodating structures and fixed lenses can be constructed from transparent materials, even if they are illustrated as opaque in the associated figures.

Any of the features of the intraocular lens systems described herein may be combined with any of the features of the other intraocular lenses described herein and vice versa. Additionally, several specific examples of embodiments in accordance with the present technology are set forth below in the following examples.

### EXAMPLES

Several aspects of the present technology are set forth in the following examples.
1. An accommodating intraocular lens assembly, comprising:
   a fixed power lens; and
   a base defining (i) an adjustable power lens and (ii) a lens-receiving volume configured to receive the fixed power lens and to permit rotation of the fixed power lens relative to the base,
   wherein an optical power of the adjustable power lens changes in response to the rotation of the fixed power lens relative to the base that causes fluid displacement within the base.
2. The accommodating intraocular lens assembly of example 1 wherein the base is configured such that (i) rotation of the fixed power lens in a first direction causes an increase to the optical power of the adjustable power lens and (ii) rotation of the fixed power lens in a second direction opposite the first direction causes a decrease to the optical power of the adjustable power lens.
3. The accommodating intraocular lens assembly of example 1 or 2 wherein:
   before the rotation of the fixed power lens, the adjustable power lens is adjustable through a first range of optical powers including a first minimum optical power and a first maximum optical power; and
   the rotation of the fixed power lens, the adjustable power lens is adjustable through a second range of optical powers including a second minimum optical power and a second maximum optical power.
4. The accommodating intraocular lens assembly of example 3 wherein the second minimum optical power is greater than the first minimum optical power.
5. The accommodating intraocular lens assembly of example 3 or 4 wherein the second maximum optical power is greater than the first maximum optical power.
6. The accommodating intraocular lens assembly of any of examples 1-5 wherein:
   before the rotation of the fixed power lens, the adjustable power lens is adjustable through a first range of depths of focus including a first minimum depth of focus and a first maximum depth of focus; and
   after the rotation of the fixed power lens, the adjustable power lens is adjustable through a second range of depths of focus including a second minimum depth of focus and a second maximum depth of focus.
7. The accommodating intraocular lens assembly of example 6 wherein the second minimum depth of focus is greater than the first minimum depth of focus.
8. The accommodating intraocular lens assembly of example 6 or 7 wherein the second maximum depth of focus is greater than the first maximum depth of focus.
9 The accommodating intraocular lens assembly of any of examples 1-8 wherein:
   the base includes an inner wall, an outer wall, a first fluid reservoir at least partially between the inner wall and the outer wall, and a second fluid reservoir positioned inwardly from and in fluid communication with the first fluid reservoir, the second fluid reservoir at least partially defining the adjustable power lens, and
   the base is configured such that rotation of the fixed power lens causes at least a portion of the inner wall to move toward the outer wall to cause fluid flow from the first fluid reservoir toward the second fluid reservoir.
10.The accommodating intraocular lens assembly of example 9 wherein:
   the portion of the inner wall includes an actuator portion,
   the fixed power lens includes a lens portion and a tab extending radially outwardly from the lens portion, and
   rotation of the fixed power lens causes the tab to contact the actuator portion and results in a deflection of the actuator portion, thereby driving fluid flow from the outer wall toward the inner wall.
11. The accommodating intraocular lens assembly of any of examples 1-10 wherein the lens-receiving volume includes a groove, and wherein the fixed power lens includes a lens portion and a tab extending radially outwardly from the lens portion, and further wherein the groove is configured to slidably receive at least a portion of the tab when the lens portion is positioned within the lens-receiving volume.
12. The accommodating intraocular lens assembly of any of examples 1-11 wherein:
   the base includes an outer fluid reservoir and an actuator portion,
   the adjustable power lens is at least partially defined by an inner fluid reservoir in fluid communication with the outer fluid reservoir, and
   in response to the rotation of the fixed power lens, the actuator portion of the base is configured to cause fluid flow from the outer fluid reservoir toward the inner fluid reservoir to change the optical power of the adjustable power lens.
13. The accommodating intraocular lens assembly of example 12 wherein, in response to the rotation of the fixed power lens, the actuator portion is configured to deflect radially outward toward the outer fluid reservoir to change a volume of the outer fluid reservoir and cause fluid flow between the outer fluid reservoir and the inner fluid reservoir.
14. The accommodating intraocular lens assembly of example 12 or 13 wherein, in response to the rotation of the fixed power lens, the actuator portion is configured to deflect radially outward toward the outer fluid reservoir to reduce a volume of the outer fluid reservoir and cause fluid flow from the outer fluid reservoir toward the inner fluid reservoir.
15. The accommodating intraocular lens assembly of any of examples 1-14 wherein the fixed power lens is positioned anterior to the adjustable power lens along an optical axis of the accommodating intraocular lens assembly.
16. The accommodating intraocular lens assembly of any of examples 1-15 wherein the lens-receiving volume includes a chamber having an opening, and wherein the chamber is configured to receive the fixed power lens via the opening.
17. The accommodating intraocular lens assembly of example 16 wherein the chamber is dimensioned to operably receive the adjustable power lens.
18. The accommodating intraocular lens assembly of any of examples 1-17, further comprising a cartridge structure configured to receive the fixed power lens and configured to be positionable within the lens-receiving volume of the base.
19. The accommodating intraocular lens assembly of example 18 wherein the fixed power lens is an off-the-shelf lens.
20. A method, comprising:
   coupling a fixed power lens of an accommodating intraocular lens to an adjustable lens structure of the accommodating intraocular lens; and
   selectively changing the fixed power lens to cause an adjustment to an optical power of an adjustable power lens of the adjustable lens structure.
21. The method of example 20 wherein selectively changing the fixed power lens includes rotating the fixed power lens relative to the adjustable lens structure.
22. The method of example 20 or 21 wherein selectively changing the fixed power lens includes one or both of (i) rotating the fixed power lens in a first direction relative to the adjustable lens structure to cause an increase to the optical power of the adjustable power lens and (ii) rotating the fixed power lens in a second direction opposite the first direction to cause a decrease to the optical power of the adjustable power lens.
23. The method of any of examples 20-22 wherein:
   coupling the fixed power lens to the adjustable lens structure includes positioning the fixed power lens at least partially within a lens-receiving area of the adjustable lens structure, and wherein the fixed power lens is a first fixed power lens, and
   selectively adjusting the fixed power lens to cause the adjustment to the optical power of the adjustable power lens includes—
removing the first fixed power lens from the lens-receiving area; and
replacing the first fixed power lens with a second fixed power lens,
wherein the first fixed power lens has a first width or diameter, and the second fixed power lens has a second width or diameter different than the first width or diameter.
24. The method of any of examples 20-23 wherein selectively changing the fixed power lens to cause the adjustment to the optical power of the adjustable power lens includes changing the fixed power lens to cause an increase or decrease to the optical power of the adjustable power lens.
25. The method of any of examples 20-24 wherein selectively changing the fixed power lens to cause the adjustment to the optical power includes causing fluid flow between an outer fluid reservoir of the adjustable lens structure and an inner fluid reservoir of the adjustable lens structure to cause a change to a volume of fluid within the inner fluid reservoir to thereby cause a change to the optical power of the adjustable power lens.
26. A method of implanting an accommodative intraocular lens (AIOL) within an eye of a patient, the method comprising:
positioning a base of the AIOL within a capsular bag of the eye;
coupling a fixed lens to the base:
determining a fit characteristic of the base relative to the capsular bag; and
if the fit characteristic of the base is below a threshold value, selectively changing the fixed lens to cause an adjustment to a size of the base.
27. The method of example 26 wherein selectively changing the fixed lens includes rotating the fixed lens relative to the base to redistribute fluid within the base to increase or decrease size of the base.
28. The method of example 26 or 27 wherein the fixed lens is a first fixed lens, and wherein selectively changing the fixed lens includes replacing the first fixed lens with a second fixed lens to increase or decrease the size of the base, wherein the first fixed lens has a first width or diameter and the second fixed lens has a second width or diameter different than the first width or diameter.
29. The method of any of examples 26-28 wherein determining the fit of the base relative to the capsular bag includes determining a range of adjustability of one or more optical properties of an adjustable lens of the base.
30. The method of any of examples 26-29 wherein selectively changing the fixed lens to cause an adjustment to the size of the base includes changing a range of adjustability of one or more optical properties of an adjustable lens of the base.

### Conclusion

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the AIOLs described herein may be combined with any of the features of the other AIOLs described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with AIOLs have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An accommodating intraocular lens assembly, comprising:
a fixed power lens; and
a base defining (i) an adjustable power lens and (ii) a lens-receiving volume configured to receive the fixed power lens and to permit rotation of the fixed power lens relative to the base,
wherein an optical power of the adjustable power lens changes in response to the rotation of the fixed power lens relative to the base that causes fluid displacement within the base.

2. The accommodating intraocular lens assembly of claim 1 wherein the base is configured such that (i) rotation of the fixed power lens in a first direction causes an increase to the optical power of the adjustable power lens and (ii) rotation of the fixed power lens in a second direction opposite the first direction causes a decrease to the optical power of the adjustable power lens.

3. The accommodating intraocular lens assembly of claim 1 wherein:
before the rotation of the fixed power lens, the adjustable power lens is adjustable through a first range of optical powers including a first minimum optical power and a first maximum optical power; and
after the rotation of the fixed power lens, the adjustable power lens is adjustable through a second range of optical powers including a second minimum optical power and a second maximum optical power.

4. The accommodating intraocular lens assembly of claim 3 wherein the second minimum optical power is greater than the first minimum optical power.

5. The accommodating intraocular lens assembly of claim 3 wherein the second maximum optical power is greater than the first maximum optical power.

6. The accommodating intraocular lens assembly of claim 1 wherein:
before the rotation of the fixed power lens, the adjustable power lens is adjustable through a first range of depths of focus including a first minimum depth of focus and a first maximum depth of focus; and
after the rotation of the fixed power lens, the adjustable power lens is adjustable through a second range of depths of focus including a second minimum depth of focus and a second maximum depth of focus.

7. The accommodating intraocular lens assembly of claim 6 wherein the second minimum depth of focus is greater than the first minimum depth of focus.

8. The accommodating intraocular lens assembly of claim 6 wherein the second maximum depth of focus is greater than the first maximum depth of focus.

9. The accommodating intraocular lens assembly of claim 1 wherein:
the base includes an inner wall, an outer wall, a first fluid reservoir at least partially between the inner wall and the outer wall, and a second fluid reservoir positioned inwardly from and in fluid communication with the first fluid reservoir, the second fluid reservoir at least partially defining the adjustable power lens, and
the base is configured such that rotation of the fixed power lens causes at least a portion of the inner wall to move toward the outer wall to cause fluid flow from the first fluid reservoir toward the second fluid reservoir.

10. The accommodating intraocular lens assembly of claim 9 wherein:
the portion of the inner wall includes an actuator portion,
the fixed power lens includes a lens portion and a tab extending radially outwardly from the lens portion, and
rotation of the fixed power lens causes the tab to contact the actuator portion and results in a deflection of the actuator portion, thereby driving fluid flow from the outer wall toward the inner wall.

11. The accommodating intraocular lens assembly of claim 1 wherein the lens-receiving volume includes a groove, and wherein the fixed power lens includes a lens portion and a tab extending radially outwardly from the lens portion, and further wherein the groove is configured to slidably receive at least a portion of the tab when the lens portion is positioned within the lens-receiving volume.

12. The accommodating intraocular lens assembly of claim 1 wherein:
the base includes an outer fluid reservoir and an actuator portion,
the adjustable power lens is at least partially defined by an inner fluid reservoir in fluid communication with the outer fluid reservoir, and
in response to the rotation of the fixed power lens, the actuator portion of the base is configured to cause fluid flow from the outer fluid reservoir toward the inner fluid reservoir to change the optical power of the adjustable power lens.

13. The accommodating intraocular lens assembly of claim 12 wherein, in response to the rotation of the fixed power lens, the actuator portion is configured to deflect radially outward toward the outer fluid reservoir to change a volume of the outer fluid reservoir and cause fluid flow between the outer fluid reservoir and the inner fluid reservoir.

14. The accommodating intraocular lens assembly of claim 12 wherein, in response to the rotation of the fixed power lens, the actuator portion is configured to deflect radially outward toward the outer fluid reservoir to reduce a volume of the outer fluid reservoir and cause fluid flow from the outer fluid reservoir toward the inner fluid reservoir.

15. The accommodating intraocular lens assembly of claim 1 wherein the fixed power lens is positioned anterior to the adjustable power lens along an optical axis of the accommodating intraocular lens assembly.

16. The accommodating intraocular lens assembly of claim 1 wherein the lens-receiving volume includes a chamber having an opening, and wherein the chamber is configured to receive the fixed power lens via the opening.

17. The accommodating intraocular lens assembly of claim 16 wherein the chamber is dimensioned to operably receive the adjustable power lens.

18. The accommodating intraocular lens assembly of claim 1, further comprising a cartridge structure configured to receive the fixed power lens and configured to be positionable within the lens-receiving volume of the base.

19. The accommodating intraocular lens assembly of claim 18 wherein the fixed power lens is an off-the-shelf lens.

20. A method, comprising:
coupling a fixed power lens of an accommodating intraocular lens to an adjustable lens structure of the accommodating intraocular lens; and
selectively changing the fixed power lens to cause an adjustment to an optical power of an adjustable power lens of the adjustable lens structure.

21. The method of claim 20 wherein selectively changing the fixed power lens includes rotating the fixed power lens relative to the adjustable lens structure.

22. The method of claim 20 wherein selectively changing the fixed power lens includes one or both of (i) rotating the fixed power lens in a first direction relative to the adjustable lens structure to cause an increase to the optical power of the adjustable power lens and (ii) rotating the fixed power lens in a second direction opposite the first direction to cause a decrease to the optical power of the adjustable power lens.

23. The method of claim 20 wherein:
coupling the fixed power lens to the adjustable lens structure includes positioning the fixed power lens at least partially within a lens-receiving area of the adjustable lens structure, and wherein the fixed power lens is a first fixed power lens, and
selectively adjusting the fixed power lens to cause the adjustment to the optical power of the adjustable power lens includes—
removing the first fixed power lens from the lens-receiving area; and
replacing the first fixed power lens with a second fixed power lens,
wherein the first fixed power lens has a first width or diameter, and the second fixed power lens has a second width or diameter different than the first width or diameter.

24. The method of claim 20 wherein selectively changing the fixed power lens to cause the adjustment to the optical power of the adjustable power lens includes changing the fixed power lens to cause an increase or decrease to the optical power of the adjustable power lens

25. The method of claim 20 wherein selectively changing the fixed power lens to cause the adjustment to the optical power includes causing fluid flow between an outer fluid reservoir of the adjustable lens structure and an inner fluid reservoir of the adjustable lens structure to cause a change to a volume of fluid within the inner fluid reservoir to thereby cause a change to the optical power of the adjustable power lens.

26. A method of implanting an accommodative intraocular lens (AIOL) within an eye of a patient, the method comprising:
positioning a base of the AIOL within a capsular bag of the eye;
coupling a fixed lens to the base:
determining a fit characteristic of the base relative to the capsular bag; and
if the fit characteristic of the base is below a threshold value, selectively changing the fixed lens to cause an adjustment to a size of the base.

27. The method of claim 26 wherein selectively changing the fixed lens includes rotating the fixed lens relative to the base to redistribute fluid within the base to increase or decrease size of the base.

28. The method of claim 26 wherein the fixed lens is a first fixed lens, and wherein selectively changing the fixed lens includes replacing the first fixed lens with a second fixed lens to increase or decrease the size of the base, wherein the first fixed lens has a first width or diameter and the second fixed lens has a second width or diameter different than the first width or diameter.

29. The method of claim 26 wherein determining the fit of the base relative to the capsular bag includes determining a range of adjustability of one or more optical properties of an adjustable lens of the base.

30. The method of claim 26 wherein selectively changing the fixed lens to cause an adjustment to the size of the base includes changing a range of adjustability of one or more optical properties of an adjustable lens of the base.
